(19) United States
(12) Patent Application Publication
(10) Pub. No.: US 2019/0008555 A1
(43) Pub. Date: Jan. 10, 2019

(54) Flexible Electrical Lead

(71) Applicant: Stimdia Medical, Inc., Edina, MN (US)

(72) Inventor: John O'Mahony, Plymouth, MN (US)

(73) Assignee: Stimdia Medical, Inc., Edina, MN (US)

(21) Appl. No.: 16/029,012

(22) Filed: Jul. 6, 2018

Related U.S. Application Data
(60) Provisional application No. 62/529,048, filed on Jul. 6, 2017.

(51) Int. Cl. A61B 17/34; A61N 1/05

(52) U.S. Cl. CPC A61B 17/3468; A61B 2017/3413; A61B 17/3403; A61N 1/05

(57) ABSTRACT

Systems having flexible electrical leads for use in medical procedures are described herein. The leads include one or more flexible electrodes having a relatively large surface area but with sufficient flexibility so as to allow the lead to fit within and be advanced through Tuohy, Sprotte and/or other types of non-coring needles.

### CROSS-REFERENCE TO RELATED APPLICATIONS

This application is a Utility filing claiming priority to U.S. Provisional Application No. 62/529,048, filed on Jul. 6, 2017 and entitled: “Lead Design”, the entire contents of which is incorporated herein by reference.

### FIELD OF THE INVENTION

Embodiments of this disclosure are directed to electrodes and lead designs for use in medical procedures.

Examples of leads and their uses are described in U.S. Pat. No. 9,682,235; U.S. Pat. No. 8,903,508 and U.S. Pat. No. 8,167,640; the entire contents of each being incorporated herein by reference.

### BACKGROUND

This disclosure herein relates to the placement of electrically stimulateable leads using a through the needle (TTN) approach and visibility under ultrasound. Electrically stimulateable needles visible under ultrasound are current commercially available. The lead may have one or more electrodes discloses designs that make it ultrasonically visible. The TTN approach is often limited by the size of the lead or catheter that is being deployed through the needle inner lumen and other methods such as peel away introducers and the seldinger technique may in some cases be used instead when applicable. These methods are not as useful for subcutaneous procedures where the device being deployed does not have the luxury of being pushed past the tip of the deployment device due to the presence of tissue. The ability to visualize the lead with respect to its anatomical location using ultrasound imaging is also important after removal of the needle. The needle is typically withdrawn leaving the lead in place. Knowing the location of the lead is critical after its deployment.

Solid electrodes such as cylindrical electrodes used in the areas of deep brain stimulation, spinal cord stimulation, phrenic nerve stimulation, peripheral nerve stimulation etc., suffer from the limitation that the diameter has to increase to increase surface area of the cylindrical electrode because the use of non-coring needles requires a bend in the inner lumen at the tip thereby limiting the length of the electrode which can pass through the turn radius. There are a number of non-coring needle designs such as Tuohy, Sprotte, Whitacre etc. commercially available. Each of these needles have a turn radius in the lumen path near the needle tip to prevent tissue coring and generally keep the lumen path at right angles to the plane of insertion at the needle tip. This bend in the lumen limits the length of the electrode that can pass through the needle and requires the use of larger needle diameters when larger surface area electrodes are required especially with commonly used inline cylindrical electrodes. The radius of the internal lumen bend is the limiting factor in the length of the electrode that may safely pass and typically also increases with the gauge of the needle. Unfortunately, larger needles result in an increase in trauma to the patient during the lead insertion process. There is a limitation in the maximum needle diameter that can be safely inserted subcutaneously in patients which is also a function of the surrounding anatomy and tissue. The larger the needle diameter the higher the insertion force and the less sensitivity the clinician has to feeling the surrounding tissue. This tactile feedback and the skill of the clinician are often critical to the safety of a procedure.

There is a need for a lead electrode design that overcomes these limitations and facilitates large surface area electrodes fitting through curvilinear needle paths like Tuohy, Sprotte and other such non-coring needles visible under ultrasound imaging.

### SUMMARY

The present disclosure describes systems, methods and apparatus which may be used to deploy large surface area flexible circular electrodes using small non-coring needles which are visible under ultrasound imaging after deployment. The surface area of the electrode may be independent of the diameter size of the needle being used. The new limitation being the diameter of the electrode and the ability to perform assembly of the lead. At some point the resistance of the connection wires to the electrode will be the determining factor. Using smaller diameter electrodes has the advantage of minimizes tissue trauma during lead deployment and thus increases patient safety. Puncturing a vein or artery with a small needle has significantly less impact when compared to puncturing with a large diameter needle. In one of the embodiments a helical coiled electrode design cut from a tubular electrode is used to provide flexibility with solid cylindrical rings at both ends ensuring the coil cannot unravel during flexing and remain intact during use. The helical coil electrode may be laser cut from a single tube cylinder of suitable electrode material. A further embodiment of the electrode design being it may be made from two or three separate components and welded together during lead assembly. Alternative flexible electrode cut patterns to helical coils are also envisaged.

### BRIEF DESCRIPTION OF THE DRAWINGS

PRIOR ART FIG. 1a is a side view of an echogenic electrical stimulateable Tuohy tipped needle.

PRIOR ART FIG. 1b is a detailed view of the needle end shown in FIG. 1a. from the bottom up perspective to illustrate the needle opening.

PRIOR ART FIG. 1c is a detailed view of the needle end shown in FIG. 1a. from a side perspective.

PRIOR ART FIG. 2 is a sectional view of a Tuohy tipped needle showing the dimensional characteristics of an embodiment of an electrode of imposed thereon.

FIG. 3 is a perspective close-up view of an embodiment of the disclosure comprising a lead having an electrode with a helical shaped cut.

FIG. 4a is a side view of a lead manufactured in accordance with an embodiment of the disclosure and which includes four spaced apart electrodes.

FIG. 4b is a close-up view of a portion of the lead shown in FIG. 4a.

FIG. 5a is a side view of the helical cut on a single electrode before assembly.

FIG. 5b is a front view of the assembly shown in FIG. 5a.

FIG. 6a is a side view of the helical cut on a multicomponent electrode before assembly.

FIG. 6b is a front view of the assembly shown in FIG. 6a.

FIG. 7a is a side view of an alternative helical cut on a single electrode before assembly.

FIG. 7b is a front view of the assembly shown in FIG. 7a.

FIG. 8 is a close-up view of the flexible electrode lead shown passing through the curved tip of a Tuohy tipped needle.

FIG. 9 is an ultrasound photograph of a helical coiled flexible electrode of the type shown in FIG. 8 shown passing through the tip of a Tuohy needle.

### DETAILED DESCRIPTION

In medical implantation procedures of the type described herein, it is axiomatic that the smaller the diameter of the needle the less impact it will have on surrounding tissue when it is moved through tissue. This is main reason smaller needs are preferred in IV therapy and subcutaneous injections. This miniaturization of the diameter conflicts with requirement of having a large surface area electrode to minimize the charge density on the electrode surface area. Charge densities greater than 30 μCoulombs/cm2-phase have been shown by McCreery and Shannon to cause tissue damage. The Shannon criteria constitute an empirical rule in neural engineering that is used for evaluation of possibility of damage from electrical stimulation to nervous tissue. The Shannon criteria relate two parameters for pulsed electrical stimulation: charge density per phase, D (μC/(phase·cm²)) and charge per phase, Q (μC/phase).

The surface area of a cylindrical electrode is primarily a function of both the electrode diameter and its length. Surface roughness also plays a factor and may be increased at a microscopic level to get a many fold increase in electrode surface area. Unfortunately, the benefits of utilizing this approach is quickly lost in vitro has been reported in the literature, due to biomaterial adhering the microstructures on the surface.

The example lead in this disclosure was chosen to have a diameter of 0.87 mm such that it could fit through a commercially available echogenic electrical stimulateable Tuohy tipped needle with a 1 mm internal diameter lumen. The lead diameter could be designed and modified to fit through any needle or number of inner needle diameters or lumens and this specific example is being given for illustrative purposes only. The design of the electrode on the lead was also chosen to be echogenic under ultrasound making it visible once deployed through the needle. The sharp edges of the spiral cut of the helix enhances visible under ultrasound.

PRIOR ART FIGS. 1a-1c show one such needle 100 that is tipped with a Tuohy tipped needle head 102 but could be a number of needle tipped designs such as Sprotte etc. that require the lumen of the needle to turn away, such as by having a bend 106, from the plane of insertion at the needle tip to prevent tissue coring (i.e. a non-coring needle). The needle may be supplied with a luer connector hub 101 for connection to a priming syringe etc. with an electrical connection 104 and wire 107 connected to the conductive needle tube 103. The needle tube 103 is coated with an electrically insulating material such that only the tip of the needle 105 is conductive. The needle 103 is hollow with an inner lumen 108 of 1 mm in diameter allowing fluids and device to pass through it. The needle 103 is capable of being inserted subcutaneously and may be used to identify specific nerves specific nerves or tissue using a combination of ultrasound visualization and electrical stimulation. Such systems or combinations thereof are relative common in performing local anesthesia, epidurals etc.

PRIOR ART FIG. 2 shows a cross-section of the needle 100 showing the inner 202 and outer 204 radii of curvature of the internal lumen 108. The needle 100, as shown, illustrates the limitation in the potential length of a solid tubular electrode that can pass through the inner lumen 108 bend 106 without becoming stuck. In this specific instance, a 0.83 mm diameter cylindrical electrode, represented by block 203, is limited to 1 mm in length 201 if it is to pass around the bend 106 without becoming stuck. The smaller the diameter of the electrode the longer the allowable length of the electrode that will pass through the bend and vice versa, the closer the diameter of the electrode comes to the internal diameter of the needle lumen the shorter the allowable electrode length. Any non-coring needle may be modeled as in this manner to determine the maximum length of a solid electrode with respect to the radii of curvature.

The larger the electrical current required to achieve electrical stimulation the greater the surface area of the electrode required to prevent tissue damage due to electrical stimulation. Damage caused by electrical stimulation is caused by a number of factors such as electrode materials, current shape of electrical stimulus, charge balance, irreversible Faradaic reactions etc. outlined by Merrill. In order to achieve a charge density requirement of 25 μC/cm2-phase, the electrode requires a length of 4 mm if it has a diameter of 0.87 mm when calculated using the Shannon criteria. This is too long in length to pass through curved lumen 108 of the 18G Tuohy tipped needle described in PRIOR ART FIG. 2 based upon both bench testing and computer aided design models. Electrodes longer than 0.9 mm become stuck if an attempt is made to pass the electrode through the lumen of the needle.

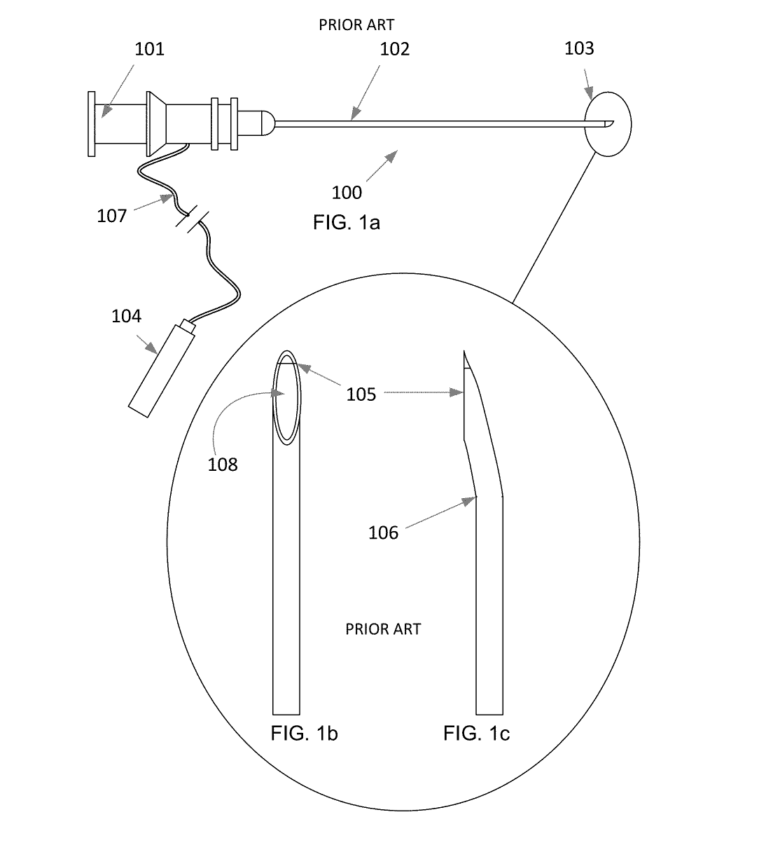

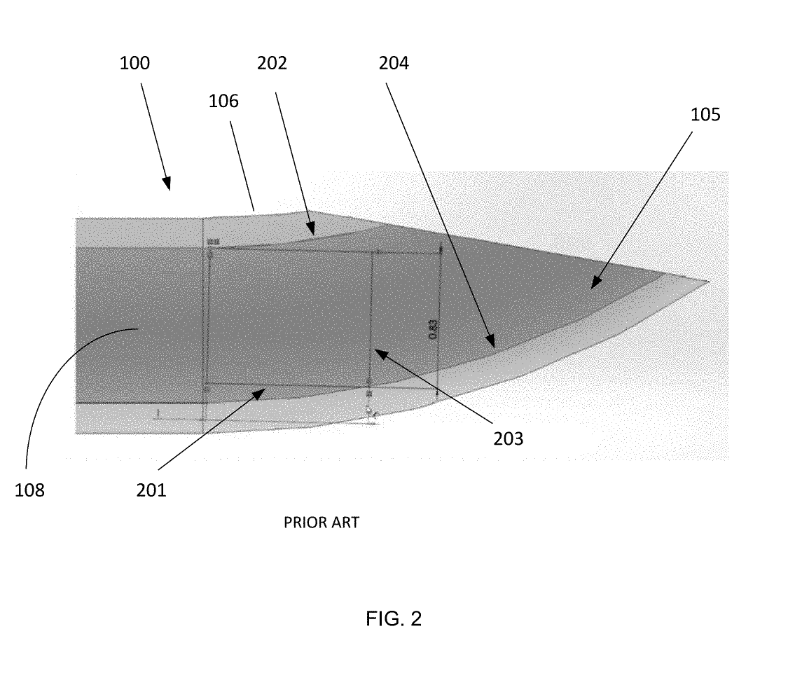

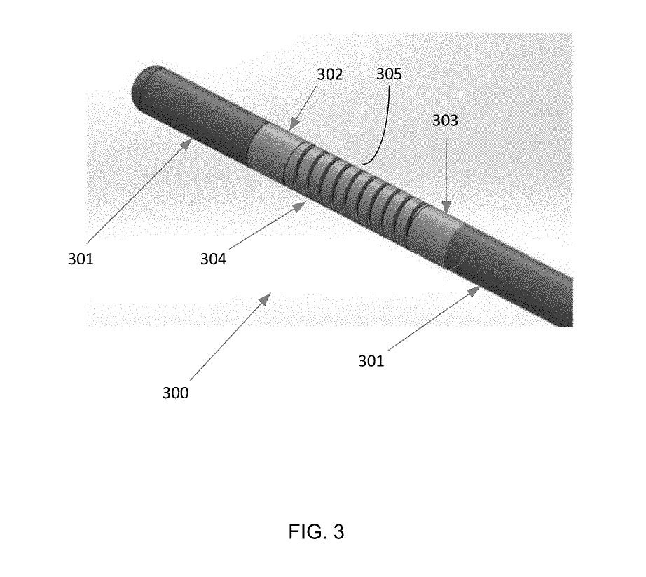

FIG. 3 shows an isometric view of an embodiment of the present disclosure which includes a lead 300 having a helical cut electrode 305, 0.87 mm in diameter and capable of passing through the needle described in PRIOR ART FIGS. 1 and 2. The lead 300 is shown manufactured from a flexible polymeric shaft 301 and electrode 305. The electrode is made from a shaft of platinum iridium but could obviously be made from another suitable metal such as platinum, gold, stainless steel, MP35 etc. Platinum iridium was chosen for its biocompatibility properties and long historical usage as a stimulating electrode material. The electrode consists of two uncut ring connectors 302 and 303 at each side of the shaft which has a helical groove 304 extending therethrough. These provide a target for the attachment of conductive wires that connect the electrode to the stimulator and allow conduction of charge. Crucially, the ring connectors 302 and 303 also prevent the helical cutting or groove 304 from unraveling by holding the ends of the helical electrode 305 in place and thus prevent the risk of sharp edges protruding causing tissue damage. Testing has shown that the use of coiled wire has this tendency or the wrapped flat wire to generate the helical coil. The use of materials such as graphene for electrodes which are known to be flexible have been proposed in the literature for electrodes but the production methods for assembly and connection of conductors have yet to be worked out and proven to be reliable.

Implanted leads are known to fail due to fatigue and the use of known tried and tested standard techniques for wire attachment to electrodes and connectors to prevent fatigue failures is key to the success of lead reliability. Two of the most common failures and causes of recall in leads are wires breaking due to fatigue or the connection to the lead becoming disconnected.

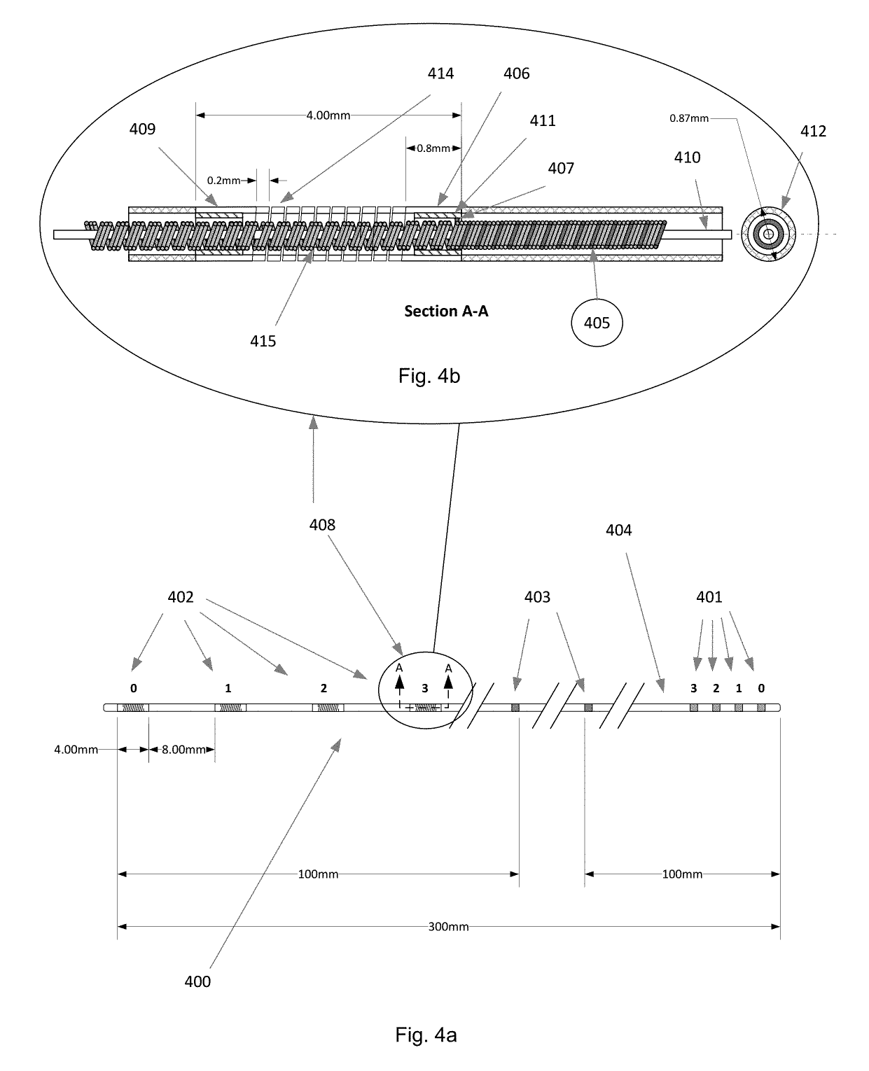

FIGS. 4a and 4b show a more detailed drawing of the overall lead 400. The example lead consists of four electrodes 402 denoted electrode 0, 1, 2 and 3 connected to the lead contacts 401 denoted 3, 2, 1, 0 by internal helical coiled wires 405.

In the embodiment shown, lead 400 may be one of several leads in communication with a stimulator which transmits an electric current to the electrodes 402 in order to stimulate a nerve or other anatomical structure. One example of a system with which the lead(s) 400 may be utilized or incorporated into is the PEPNS system described in U.S. Pat. No. 9,682,235, the entire contents of which are incorporated herein by reference.

An IS4 type connector or other such similar connector design may be used to provide connection to the contacts between the lead and the stimulator. The contacts provide electrical contacts to connect to an electrical stimulator. In this case contact 0, 401 is connected to electrode 0, 402 and contact 1 is connected to electrode 1 and so on. There are 4 electrodes 402 in this lead but many additional electrodes are possible using the configuration shown. The lead is supplied with marker bands 403 spaced at 100 and 200 mm intervals in the 300 mm length lead. The marker bands are used to help the user identify the length of the lead inserted into the patient. The lead body material between the leads, marker bands and contacts is made from a transparent polyurethane polymer providing flexibility and encapsulating the internal wires and electrical connections. A cross-section of one of the leads is shown in section A-A 408 along with the side view 412.

Coiled wires 405 have been historically used where flexibility and fatigue resistance are required. This coiled design also prevents the connections from coming under strain when the lead is under tensile forces. The number of individual wires coiled is a function of the number of electrodes used. In this case 4 coiled wires 405 are wound in parallel and each wire is connected to a contact 401 and electrode 402. The wires may be made from silver filled MP35N to minimize electrical resistance and provide maximum strength and fatigue resistance. The wire 405 is coated with an insulating material such as ETFE which is a copolymer of tetrafluoroethylene (TFE) and ethylene to prevent electrical shorting between wires. In section A-A 408 the connection of electrode 3 and the wire is shown as a swage crimp connection between the electrode ring 406 and the swage ring 411. The wire 407 which contains four wires, one for each electrode contains only 3 wires after it exists the electrode 415. Laser or electrical welding of the wire 407 to the ring electrode 406 are also possible. The ring connector 406 also provide an ideal area for this connection. The ring connectors, 406 and 409 were designed to be 0.8 mm in length and the helical laser cut electrode width to be 0.2 mm 414 with gaps of 05 mm between each helical ensuring the electrode can flex as it passed through the needle tip. These ratios may be varied depending upon the electrode flexibility required, the lead tensile strength requirements and the radius of the needle bend.

Electrode flexibility is achieved by cutting a helical shape in to the electrode using a metal laser cutter. The length of the uncut electrode and width of the helical cuts have to be small enough to fit through the curved needle tip and flexible enough to allow the electrode to bend. The smaller the distance between the helical cuts the weaker the electrode is in terms of tensile strength but the lower the force required to pass through the needle. The wall thickness of the electrode is approximately 08 mm.

During lead insertion, a stylet 410 may be used to stiffen the lead. A stylet retention endcap made of MP35N may be used to prevent the stylet wire from perforating the end of the lead and causing patient harm. Alternatively, the stylet 410 may be manufactured as part of the lead and be used to increase the tensile strength of the lead and be glues in place. The smaller the cross-sectional area of the lead the lower its tensile strength will be. A nitinol stylet may be used to increase tensile strength while improving the leads ability to return back to its original shape. Testing showed that tensile strength could be increased to >9N with less than 20% elongation over 1 minute versus tensile strength was between 4.5 to 5N under the same test conditions without the use of a 6 thou nitinol stiffening member.

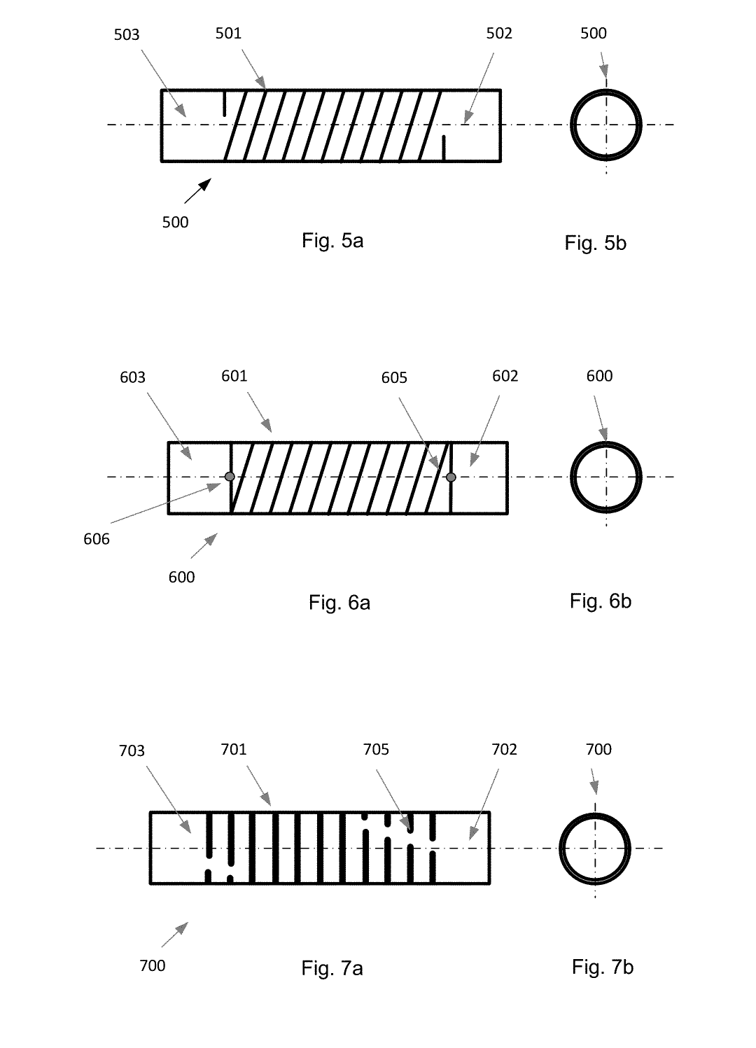

FIGS. 5a and 5b shows the cylindrical electrode 500 with a laser cut helical profile on the electrode 501. The diagram shows the electrode 500 in elevation and side view. The helical groove 504 is cut from a solid electrode tube leaving ring electrodes spaces 502 and 503 on either end of the electrode body without helical cuts.

FIGS. 6a and 6b shows an alternative manufacturing option of using 3 components to manufacture the electrode 600. Two ring electrodes 602, 603 may be connected to a helical coil 601. The ring electrodes and helical coils may be welded together as shown in welds 605 and 606. The helical coil 601 may be laser cut or may be wound on a mandrel. The benefit of welding the helical coil to the ring electrodes is that it prevents the coil from unwinding and keeps the assembly together during manufacturing.

There are many different shapes that can be cut into the electrode that will provide adequate flexibility and ultrasound visibility. FIGS. 7a and 7b shows an alternative way to cut the electrode 700 to providing adequate flexibility. In this case the cuts are perpendicular to the axis and the part left uncut spirals 705 as a helix. Two areas of the electrode702 and 704 are left uncut to prevent the helix from potential unraveling.

FIG. 8 shows a photograph of a helical coiled flexible electrode 801 passing through the tip of a Tuohy needle 800 with echogenic indentations. The transparent polyurethane tubing 802 containing the helical coil wire and a stiffening member 803. Stiffening member 803 may be any type of material suitable for use as a stiffening member for assisting in the advancement of the lead and support of the electrode. In at least one embodiment, the stiffening member is a wire, braid of wires forming a member constructed of Nitinol. Using this or similar design of stiffening member with any of the embodiments shown or described herein, it is possible to pass any appropriately sized surface area electrode through a curved needle path.

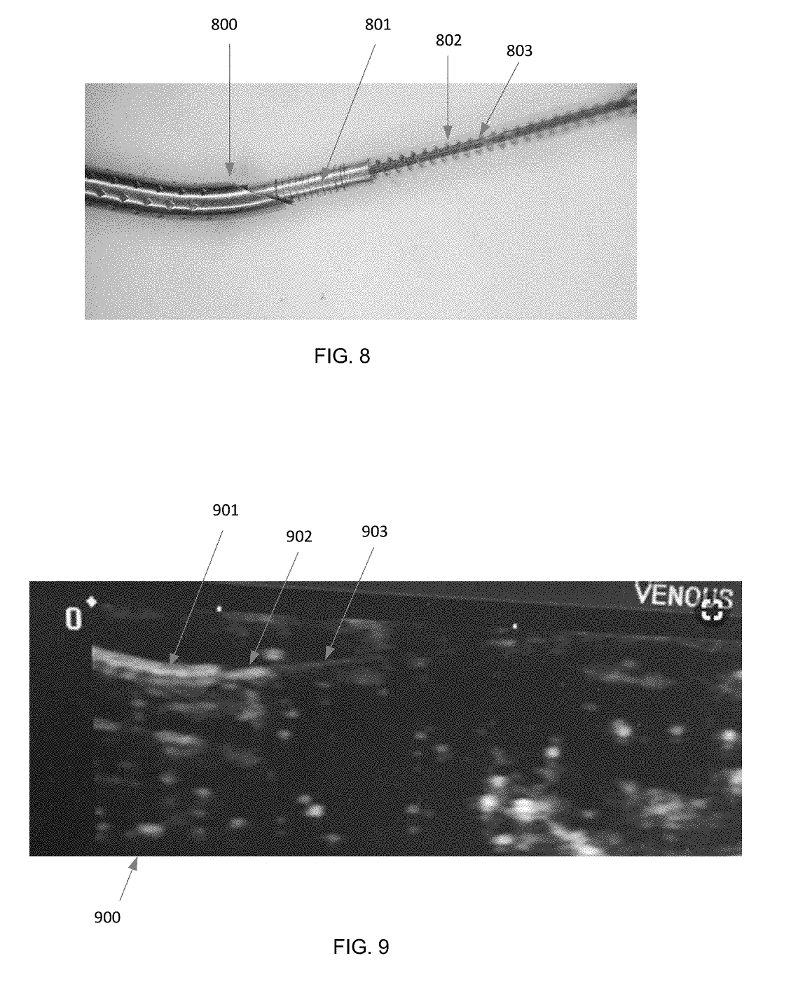

FIG. 9 shows an ultrasound photograph 900 of a helical coiled flexible electrode 902 passing through the tip of a Tuohy needle 901 with echogenic indentations in the same configuration as sown in FIG. 8. The electrode 902 is clearly more visible that the polyurethane tubing 903 which is moving out of the ultrasound plane. The electrode 902, needle 901 and lead body 903 as shown in FIG. 9 with ultrasound imaging are the same lead as shown in the photograph in FIG. 8 electrode 800, needle 800 and lead body 802 respectively.

### REFERENCES

In addition to the details and descriptions provided above, the following publications should be considered as part of the present disclosure.

Merrill D R, Bikson M, Jefferys J G. Electrical stimulation of excitable tissue: design of efficacious and safe protocols. J Neurosci Methods. 2005 Feb. 15;141(2):171-98. Review. PubMed PMID: 15661300; the entire contents of which are incorporated herein by reference.

McCreery D B, Agnew W F, Yuen T G H, Bullara L. “Charge density and charge per phase as cofactors in neural injury induced by electrical stimulation,” IEEE Trans. Biomed. Eng., vol. 37(10):996-1001; the entire contents of which are incorporated herein by reference.

McCreery D B, Agnew W F, Yuen T G H, Bullara L. “Comparison of neural damage induced by electrical stimulation with faradic and capacitor electrodes,” Ann. Biomed. Eng., vol. 16(5):463-81; the entire contents of which are incorporated herein by reference.

Shannon R V “A model of safe levels for electrical stimulation.” Biomedical Engineering, IEEE Transactions 39: 424-426; the entire contents of which are incorporated herein by reference.

The many features and advantages of the invention are apparent from the above description. Numerous modifications and variations will readily occur to those skilled in the art. Since such modifications are possible, the invention is not to be limited to the exact construction and operation illustrated and described. Rather, the present invention should be limited only by the following claims.

What is claimed is:

1. A system comprising at least one flexible electrode lead and a non-coring implantation needle, the non-coring implantation needle defining a lumen, the lumen having at least one bend, the at least one flexible electrode lead being moveable through the lumen and passing across the at least one bend, the at least one flexible electrode lead comprising an elongate shaft, a stiffening member and a plurality of tubular electrodes disposed about the elongate shaft, each of the plurality of tubular electrodes comprising pair of rings and a shaft extending between the rings, the shaft defining a helical groove therethrough.

2. The system of claim 1, wherein the stiffening member is a nitinol wire.
